(19) United States
(12) Patent Application Publication
(10) Pub. No.: US 2007/0225688 A1
(43) Pub. Date: Sep. 27, 2007

(54) Urinary catheter device

(75) Inventor: Mary Elizabeth Goodwin, DeWitt, AR (US)

Correspondence Address: PATRICIA A. SWEENEY, 1835 PLEASANT ST., WEST DES MOINES, IA, 50265, US

(73) Assignee: ARKANSAS STATE UNIVERSITY, State University, AR (US)

(21) Appl. No.: 11/686,562

(22) Filed: Mar. 15, 2007

Related U.S. Application Data
(60) Provisional application No. 60/785,302, filed on Mar. 23, 2006.

(51) Int. Cl. A61M 27/00

(52) U.S. Cl. 604/544; 604/322; 604/327

(57) ABSTRACT

A urinary catheter device is shown which improves on prior devices by providing for a means to lengthen and shorten the tube used to transport urine to a collection receptacle. A transport tube is provided which is connected to the catheter or in fluid direct communication with the ureters where a stoma opening is instead provided. The transport tubing includes a contraction region, which can in an example be crests and groves or a helical coil, which allows for the region to be contracted during movement, transport or the like of the patient.

### REFERENCE TO RELATED FILINGS

This application claims priority to previously filed and co-pending application U.S. Ser. No. 60/785,302 filed Mar. 23, 2006, the contents of which are incorporated in their entirety.

### BACKGROUND

The invention described herein is to an animal urinary collection device which provides for ease in mobility and transport of the animal or patient when connected to the device, by providing for a means to lengthen and shorten the tube used to transport the urine to a collection receptacle.

In general, in a number of medical and veterinary catheterization procedures, urine exits the animal or patient through a medical device inserted into the animal. Such devices typically provide for a catheter, which is a tube that is inserted into the bladder to withdraw urine from the bladder. An aperture, membrane, or other means of allowing the urine to pass from the distal end through and out the proximal end is provided. Often there are one or more apertures at each end of the catheter. This tube allows for the passage of urine from the bladder through the tube. The catheter is then connected with a tube which drains from the catheter and into a collection device. These types of devices are well known to one skilled in the art and include those described at WO 91/00074; U.S. Pat. No. 3,374,790 and catheters described at U.S. Pat. Nos. 4,277,533; 3,432,865 and 4,270,213. (All references cited are incorporated herein by reference.) A number of adaptations for these devices have been described, including adaptations to the catheter device for sealing insertion of the catheter (WO 91/00074); for disposable self-lubricating catherization devices which aid in introduction of the devices into the urethral opening (U.S. Pat. No. 6,090,075); a guiding catheter (U.S. Pat. No. 6,858,024); urine collection bags for such devices (U.S. Pat. No. 6,887,230); and devices improved for temporary catheterization. When used as a temporary catheterization, the catheter is used alone as a means of voiding the bladder, and after use, is removed. Such temporary catherizations are used in situations where the patient may suffer from conditions such as urinary retention, the inability to evacuate urine, obtaining a sterile urine specimen in a doctor's office, or for convenience when it is not otherwise possible to evacuate the bladder. Examples of such temporary catheter devices include those described at U.S. Pat. No. 6,852,098; U.S. Pat. No. 6,090,075; and WO 91/00074. By way of example, Byrne in U.S. Pat. No. 6,852,098, describes a catherization device intended for transient catheterization in females. It includes a catheter having a distal portion for insertion into the urethra, which includes a rigid, thin walled tube with corrugated region for flexing. This device is used for ease of insertion in that it can be bent and is flexible, allowing manipulation of the device during insertion. It can also be used in bending the catheter portion up against the body without buckling of the tube.

In a further common application in medical settings, in addition to providing for voiding urine, urine collection is required and a closed system is employed. In hospitals and nursing homes, for example, patients may be catheterized long term, and the need arises to have the catheter connected to a transport tube (sometimes referred to also as a drainage tube or hose), which is then connected to a collection device. The urine flows out from the bladder through the catheter, then through the transport tube and into a urine collection bag.

In a variation on such devices, a urostomy may be performed and used in connection with urine draining systems. This may be required where the bladder must be removed, as when bladder cancer occurs. In this instance, an ileal conduit is formed using a six to ten inch segment of the ileum or small intestine. One end is closed with stitches or staples, and the other brought to the surface of the abdomen and forms a stoma. Ureters are implanted into the closed end of the conduit and urine passes from the ureters into the stoma and into a urine collection device. Such devices may also include a transport tube which allows passage of urine into a urine collection device.

The transport tube is typically flexible tubing, allowing for it to be easily moved when the patient is sitting or lying. Such transport tubes are commonly long, in order to avoid accidentally pulling the catheter out of the patient, allowing the bag to be connected to a pole, to a wheelchair or the bed, and to keep it out of the way and to avoid the weight of the bag from pulling on the catheter. There are many variations employed by manufacturers of such devices in the length of the tubing of the entire catheter device. Commonly, when used in hospitals, extended care facilities, or other nursing facilities, the tubes may be about one to four or more feet in length. The transport tubing, while providing certain advantages, causes considerable disadvantage when the patient is mobile. Whether moving from one point to another in a wheelchair, walking with the bag connected to a pole, or the like, the tubing must be held or hung from the chair or pole. Entanglement in the wheels of the chair is a risk, as is tripping on the tubing, or catching the tubing such that the catheter is accidentally pulled out. To ease movement from a wheelchair to a bed, for example, at times the collection bag may be placed on the floor so the patient can move without the bag and tubing restricting mobility; however, this is not sanitary. Alternatively, the patient or individual assisting the patient, may hold the tubing or bag when the patient is mobile.

The present invention overcomes these problems by providing a means to temporarily shorten the length of the transport tubing.

### SUMMARY OF THE INVENTION

A means to temporarily shorten a transport tubing section of a urine collection device is described which provides for a contraction region of the transport tube, which tube is in fluid communication with a catheter on one end and a urine collection device on the other end. In one embodiment, the contracting region is a corrugated region of crests and grooves. An embodiment provides the corrugated region may further be retained in a compressed condition by use of a retaining device, such as a clamp. In another embodiment, the contracting region is a helically coiled region. Yet another embodiment provides the contracting region may be corrugated and helically coiled. Further embodiments provide for a sheath surrounding at least a portion of the transport tubing. The sheath may include a retaining device, such as overlapping notches or an annular ring which slips over a bulge on the transport tube.

### DESCRIPTION OF THE DRAWINGS

FIG. 1 is a schematic of a catheterization device.

FIG. 2 is an elevation view of an embodiment of the transport tube of the invention.

FIG. 3 is a partial side planar view of a transport tube of the invention showing corrugated and convoluted contraction regions having crests and ridges.

FIG. 4 is a partial side sectional view of a contraction region of the invention in compressed and non-compressed state, and having a sheath.

FIG. 5 is a partial side sectional view of the contraction region fully compressed and having a sheath with a locking junction.

FIG. 6 is a partial side planar view of a helically coiled transport tube of the invention.

FIG. 7 is a partial side sectional view of a contraction region of the transport tube and a sheath.

FIG. 8 is a partial side sectional view of a transport tube with a contraction region and a sheath in two sections having stop notches.

FIG. 9 is a partial side sectional view of a helically coiled contraction region having a sheath with a conical cap.

### DESCRIPTION OF A PREFERRED EMBODIMENT

In the present invention an improvement to urine collection devices is provided.

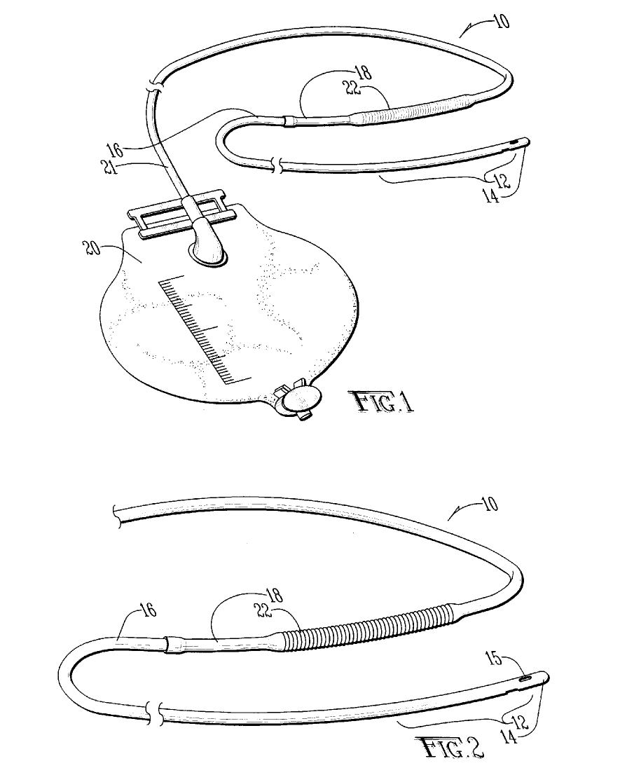

The entire catheterization device 10 is represented in FIG. 1. The catheter tube 12 has a distal end 14 inserted into the urethra, and a proximal end 16 connected to the transport tube 18, which links drainage of the fluids to the collection bag 20, which commonly is detachably attached to a pole or similar device, or held by the patient or attendant. When the patient must be transported to another location, the transport tube 18 is often an obstacle in that it can become entangled in a wheelchair, mobile bed or the like. Such tubes are often long, commonly about four feet in length, to allow medical personnel to move freely about the patient without disrupting the drainage systems. Here, the transport tube can be shortened. This is provided by a contraction section within the transport tube that can be shortened during transport.

In one embodiment of the invention, the catheter tube 12 is at least about one to two centimeters longer than the length of the urethra. In one example, since the length of the female urethra is normally about 4 centimeters, the catheter tube is preferably about five to seven centimeters, or longer. The outer diameter of the tube varies, and could be narrower in diameter than the urethra, approximately the same diameter, or larger in diameter than the urethra. The tissue will form around the catheter, and in some instances larger diameter may be desired when stretching of tissue results from ongoing catheterization. Clearly, there are many variations upon the length and specifics of the catheter portion inserted into the patient. In some embodiments, a balloon may be provided in the distal portion of the catheter, which may be inflated after insertion into the bladder, and maintains the catheter in place. Where a balloon is included, an inlet port and passageway would also be provided in the catheter 12 allowing water to be injected into the passageway and into the balloon, inflating it. Further, where an urostomy has been performed, a catheter may not be used, and instead urine passes from the ureters in the stoma to a transport tube and into a collection device.

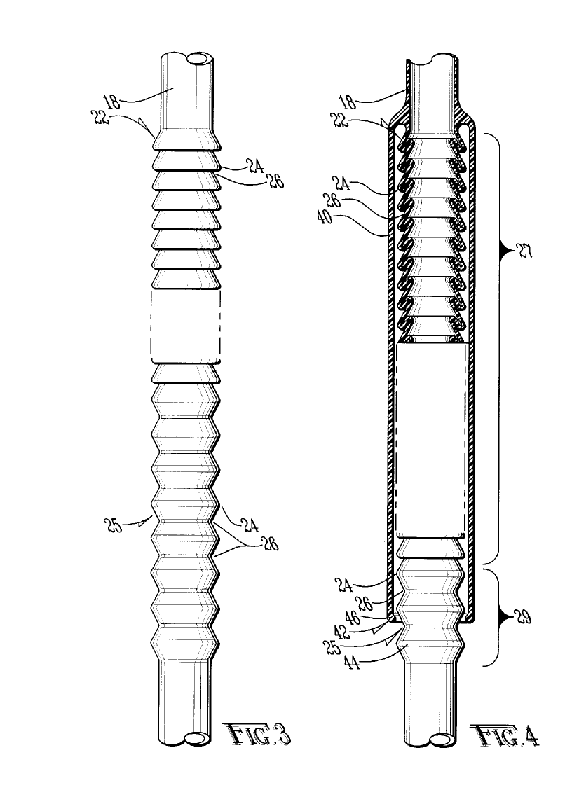

The transport tubing 18 is in sealing fluid connection to the proximal end 16 of the catheter tube and at its terminal end 21 is in sealed fluid connected with the collection bag 20. FIG. 2 shows the catheter tube 12 having an aperture 15 at its distal end for passage of urine from the bladder into the catheter. It is understood by one skilled in the art that the catheter tube 12 and the transport tubing 18 may form one continuous tubing, or may involve separate tubing sections in sealed fluid connection to one another. The device may or may not include one or more valves which prohibit back-flow of the urine and may be included in the catheter and another valve included at the upper end of the collection device. Optionally, a valve may be included at the lower end of the collection device, preferably with a drainage pipe, such that the container can be emptied when desired. At least one contraction region 22 is provided in transport tube 18. The contraction region 22 is placed sufficiently distant from the patient body in transport tubing 18 to aide in ready manipulation of the region by the patient or another individual. In one embodiment, the region is at least about 20 or more centimeters from the insertion point. There may be more than one contraction region provided along the length of the transport tube.

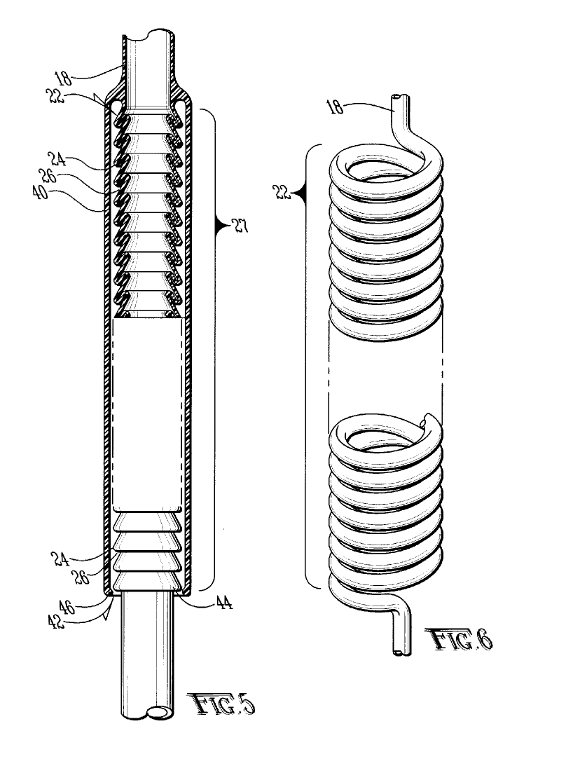

In an embodiment of the invention, as shown in FIG. 3, the contraction region 22 is a corrugated region, providing for crests 24 and grooves 26 in the tube. Certain manufacturers at times also refer to such tubing as convoluted, particularly where the crests 22 and grooves 24 are more rounded as shown at 25 in FIG. 3. The crests and grooves allow the contraction region 22 to be compressed axially along its length, as can be seen in FIGS. 4 and 5. In FIG. 4, the contraction region is shown at 27 with the crests and grooves compressed, and the non-compressed state shown at 29. In FIG. 5 the contraction region 22 is shown fully compressed. As is evident, this allows for the length of the transport tube 10 to be shortened or lengthened.

The material used in the catheter and in the transport tubing varies among manufacturers, and is not critical to the invention, provided it allows for the contraction region or regions to be formed in the transport tubing. For example, polyvinylchloride, polyethylene, polyurethanes, silicones, and polypropylene, which compositions are capable of being sterilized, can be extruded into desired shapes and are durable. Corrugated regions can be readily formed in such material. An example of one type of corrugated tubing which may be employed in the invention is that produced by the company TexLoc™ in Forth Worth, Tex. (www.texloc.com) and which goes by the name of Tex-Flex™. It is made from fluorinated ethylene propylene or perfluoroalkoxy, is capable of being exposed to high temperatures and has a non-stick surface. There are many manufacturers of such tubing, such as Tef-Cap of West Chester, Pa. (www.tefcap.com) and Zeus® Industrial Products of Orangeberg, S.C., among others.

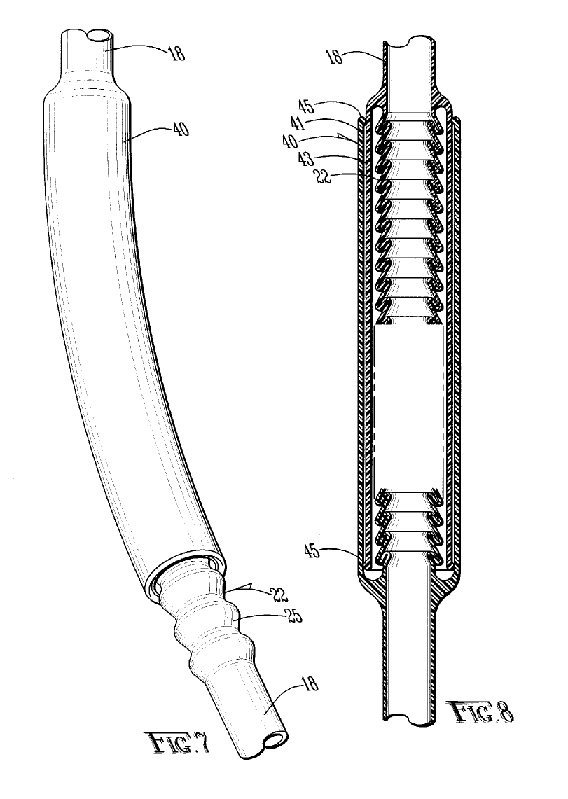

In another embodiment of the invention, the transport tubing 18 may itself be coiled as shown in FIG. 6. A helical coil allows for expansion and contraction of the tubing. For example, Zeus® (www.zeusinc.com), produces retractable coil tubing from fluoropolymer resins, including fluorinate ethylene propylene; polytetrafluoroethylene and polytetrafluoroethylene-perfluoromethylvinylether; ethylene tetrafluoroethylene; and polyvinylidene fluoride, among others. The company uses a manufacturing process to “heat set” fluoropolymer tubing into the helical coil. This allows for increased retraction and flexibility. In a further variation, the corrugated region may be both corrugated and coiled helically. Both Zeus® and Tef-Cap produce such tubing which they term corrugated, coiled, or convoluted.

The transport tubing 18 may optionally be covered by a sheath 40, shown in FIG. 7. The sheath provides several advantages, in that it can aid in keeping the transport tubing clean, prevent dirt, hair and other particles from being trapped in either the crests or grooves of the contraction region, and, particularly, as shown in FIG. 9, when used with a helical coil, prevents entanglement with other objects. The sheath can cover all or a portion of the transport tubing.

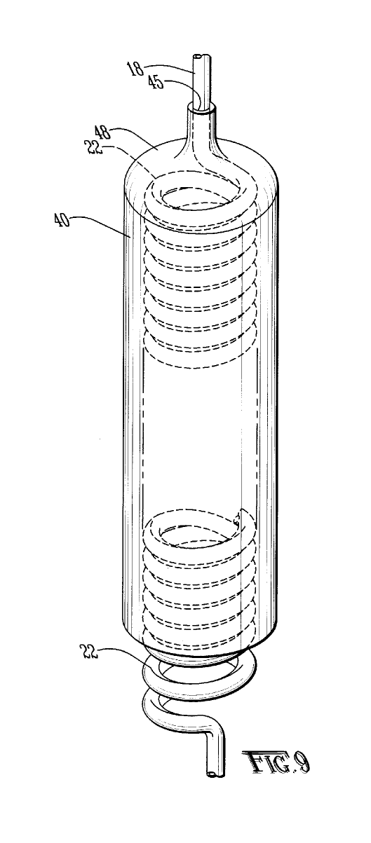

When the transport tube is of sufficient rigidity, the contraction region will remain compressed. If the tube is less rigid, another device may be incorporated to assist in keeping the contracting region compressed. It is appreciated that any number of devices may be useful in retaining the compressed condition of the contracting region, such as, for example, a clamp, spring, hook-and-eye type device such as Velcro®, elastic band, a cord attached to a clamp, or other retaining device. FIG. 5 shows one option for such a retaining device. Here, transport tube 18 includes a locking junction 42 comprising a bulge 44 at one end of the contraction region 22 of transport tubing and an annular ring 46 at one end of the sheath 40 which aligns with the bulge 44. This bulge can comprise one of the crests 24 or a separate outwardly extending member. FIG. 4 also shows a section view with annular ring 46. As in FIG. 5, the ring 46 is designed to slip over and hold in place the bulge 44. Thus, when compressing contraction region 22, the bulge 44 is slipped into the ring 46, which then holds the bulge 44 in place, keeping the contraction region 22 compressed. In another embodiment, as in FIG. 8, the sheath 40 is in two sections, a first section 41 and second section 43. Notches 45 protrude interior to the sheath 40 and are provided at each coextensive terminal end of the first and second sections. The notches 45 align such that the first section 41 is slipped over the second section 43 of the sheath and the stop notches 45 engage to prevent the two sections from pulling apart. In yet another variation at FIG. 9, the sheath 40 surrounds the helically coiled contraction section 22 of transport tube 18 and includes at either end a conically shaped cap 48. When compressing laterally the contraction section 22, the contraction section is fed into the cap 48, where it expands and is prevented from slipping out of sheath 40 by virtue of the conical shape of the cap and the expansion of the coil to a width greater than the aperture 45 of the cap 48. When a helical coil contraction region is used, it may alternately be desirable to use a device to retain the coil in an extended position when not in transport, allowing the coil to revert to its coiled condition during transport of the patient. Clearly, many variations are available to retain the shape of the transport tube in either an extended or compressed state.

What is claimed is:

1. A urinary catheter device comprising:
(i) a catheter tube for at least partial insertion into a urethra of an animal, comprising a tube with a distal region and proximal region, the tube capable of allowing passage of urine through the tube;
(ii) a transport tube connected to the proximal region of the catheter tube allowing transport of urine from the catheter tube to the transport tube, the transport tube comprising at least one contraction region capable of extension and compression such that the transport tube may be expanded or retracted by extension or compression of the contraction region; and
(iii) a urine collection receptacle connected to the transport tube.

2. The device of claim 1 wherein the contraction region comprises crests and grooves.

3. The device of claim 1 wherein the contraction region comprises at least one helical coil.

4. The device of claim 3 wherein the at least one helical coil is corrugated.

5. The device of claim 1 further comprising a retaining device to releasably retain the contraction region in a retracted state.

6. The device of claim 5 wherein the retaining device is selected from the group consisting of a clamp, spring, hook-and-eye device, or elastic band.

7. The device of claim 1 further comprising a sheath surrounding at least a portion of the contraction region.

8. The device of claim 7 wherein the sheath comprises a retaining device releasably retaining the contraction region in a retracted state.

9. The device of claim 1 wherein the transport tubing is produced from material of sufficient rigidity that manual compression of the contraction region allows the region to be retained in a compressed state.

10. The device of claim 9 wherein the transport tubing is produced from material consisting of polyethylene, polyurethanes, silicone and polypropylene.

11. The device of claim 1 wherein the contraction region is at least about 20 cm distal to the urethra of the animal.

12. A urinary catheter device comprising:
(i) a catheter tube for at least partial insertion into the urethra of an animal, comprising a tube with a distal region and proximal region, the tube capable of allowing passage of urine through the tube;
(ii) a transport tube connected to the proximal region of the catheter tube allowing transport of urine from the catheter tube to the transport tube, the transport tube at least about 20 centimeters from the urethra
(iii) a contraction region selected from the group consisting of crests and grooves region, and at least one helically coiled region, the contraction region capable of extension and compression such that the transport tube may be expanded or retracted by extension or compression of the contraction region;
(iv) a retaining device releasably retaining the contraction region in a contracted state; and
(v) a urine collection receptacle connected to the transport tube.

13. A urine collection device comprising a transport tube in fluid connection to ureters in a patient allowing transport of urine from the ureters through the transport tube, the transport tube comprising a least one contraction region capable of extension and compression such that the transport tube may be expanded or retracted by extension or compression of the contraction region, and a urine collection receptacle connected to the transport tube.

14. The device of claim 13 wherein the contraction region comprises crests and grooves.

15. The device of claim 13 wherein the contraction region comprises at least one helical coil.

17. The device of claim 13 further comprising a sheath surrounding at least a portion of the contraction region.

18. The device of claim 13 wherein the device further comprises a catheter tube for at least partial insertion into the urethra of a patient, the urethra leading to the ureters, and the catheter in fluid communication with the transport tube.
